(19) United States
(12) Patent Application Publication
(10) Pub. No.: US 2026/0158145 A1
(43) Pub. Date: Jun. 11, 2026

(54) QUINOLINE DERIVATIVE-CONTAINING PHARMACEUTICAL COMPOSITION

(71) Applicant: Eisai R&D Management Co., Ltd., Tokyo (JP)

(72) Inventor: Masashi Bando, Kakamigahara (JP)

(21) Appl. No.: 19/408,622

(22) Filed: Dec. 4, 2025

Related U.S. Application Data
(63) Continuation of application No. 17/228,025, filed on Apr. 12, 2021, now Pat. No. 12,508,313.
Application No. 17/228,025 is a continuation of application No. 13/923,858, filed on Jun. 21, 2013, now abandoned.
Application No. 13/923,858 is a continuation of application No. 13/322,961, now abandoned.

(30) Foreign Application Priority Data
Aug. 19, 2009 (JP) P2009-190145

(51) Int. Cl. A61K 47/02; A61K 9/16; A61K 9/48; A61K 31/47; C07D 215/48

(52) U.S. Cl. CPC A61K 47/02; A61K 9/1611; A61K 9/1652; A61K 9/485; A61K 9/4866; A61K 31/47; C07D 215/48

(57) ABSTRACT

A pharmaceutical composition comprising a compound represented by the formula (I) or pharmaceutically acceptable salt thereof or solvate thereof, and a basic substance is excellent in dissolution, is stable even after a long term storage, and is useful as a preventive or therapeutic agent against a tumor:

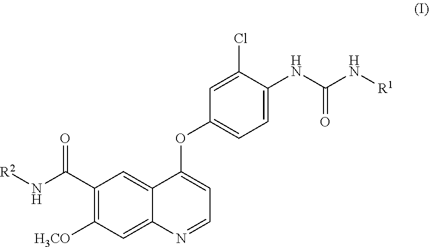

wherein, R¹ is a hydrogen atom, a C₁₋₆ alkyl group or a C₃₋₈ cycloalkyl group; and R² is a hydrogen atom or a methoxy group.

### TECHNICAL FIELD

The present invention relates to a pharmaceutical composition comprising a quinoline derivative, useful as a medicament. More specifically, the present invention relates to a pharmaceutical composition improved in dissolution of a quinoline derivative or a pharmaceutically acceptable salt thereof or a solvate thereof.

### BACKGROUND ART

A quinoline derivative represented by the formula (I) or a pharmaceutically acceptable salt thereof or a solvate thereof (hereinafter referred to as quinoline derivative (I)) has been known to have a potent angiogenesis inhibitory effect (Patent Literature 1) and a c-Kit kinase inhibitory effect (Patent Literature 2) and to be useful as a preventive or therapeutic agent against various tumors such as thyroid cancer, lung cancer, melanoma and pancreatic cancer, and as an metastatic inhibitor against these tumors:

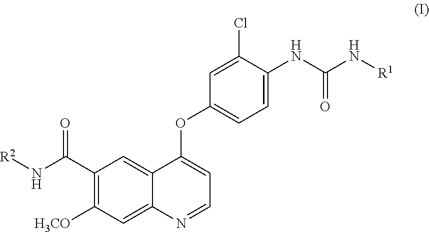

wherein, R¹ is a hydrogen atom, a C₁₋₆ alkyl group or a C₃₋₈ cycloalkyl group; and R² is a hydrogen atom or a methoxy group.

However, the quinoline derivative (I) has been found to degrade under humidifying and warming storage conditions when formulated into a pharmaceutical composition. In addition, when the pharmaceutical composition absorbs moisture, dissolution of the quinoline derivative (I) from the pharmaceutical composition that is an active ingredient may delay because of gelation on the surface of the composition. In order to overcome these problems, a pharmaceutical composition which includes the quinoline derivative (I), (1) a compound, a 5% (w/w) aqueous solution or suspension of which has a pH of 8 or more, and/or (2) silicic acid, salt thereof or solvate thereof has been developed (Patent Literature 3).

### CITATION LIST

### Patent Literature

Patent Literature 1: WO 2002/32872
Patent Literature 2: WO 2004/080462
Patent Literature 3: WO 2006/030826

### SUMMARY OF INVENTION

### Technical Problem

However, development of a pharmaceutical composition further excellent in the dissolution of the quinoline derivative (I) has been desired. Thus, the present invention is aimed at providing a pharmaceutical composition that is excellent in dissolution of the quinoline derivative (I) that is maintained even after long term storage.

### Solution to Problem

The present inventors have intensively studied in order to solve the problems above and surprisingly have discovered the configuration below could solve the problems and have completed the present invention.

Specifically, the present invention provides the following <1> to <12>.
[1] A pharmaceutical composition comprising:
   (1) a compound represented by the formula (I) or pharmaceutically acceptable salt thereof or solvate thereof:

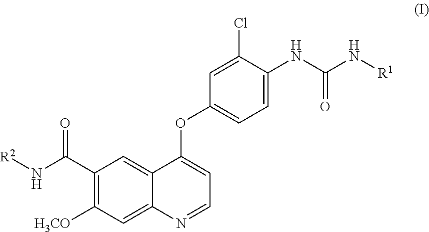

wherein R¹ is a hydrogen atom, a C₁₋₆ alkyl group or a C₃₋₈ cycloalkyl group; and R² represents a hydrogen atom or a methoxy group; and
   (2) a basic substance.
[2] The composition according to [1], wherein the basic substance is a carbonate.
[3] The composition according to [2], wherein the salt is an alkaline earth metal salt.
[4] The composition according to [3], wherein the alkaline earth metal salt is a magnesium salt or a calcium salt.
[5] The composition according to any one of [1] to [4], further comprising a disintegrating agent.
[6] The composition according to [5], wherein the disintegrating agent is carmellose sodium, carmellose calcium, carboxymethyl starch sodium, croscarmellose sodium, low-substituted hydroxypropylcellulose or crospovidone.
[7] The composition according to any one of [1] to [6], wherein R¹ is a hydrogen atom, a methyl group, an ethyl group, an n-propyl group or a cyclopropyl group.
[8] The composition according to any one of [1] to [7], wherein R¹ is a cyclopropyl group.
[9] The composition according to any one of [1] to [8], wherein R² is a hydrogen atom, a methoxy group or an ethoxy group.
[10] The composition according to any one of [1] to [9], wherein R² is a hydrogen atom.
[11] The composition according to any one of [1] to [10], wherein the pharmaceutically acceptable salt is hydrochloride, hydrobromide, p-toluenesulfonate, sulfate, methanesulfonate or ethanesulfonate.
[12] The composition according to any one of [1] to [11], wherein the compound represented by the formula (I) is 4-(3-chloro-4-(cyclopropylaminocarbonyl)aminophenoxy)-7-methoxy-6-quinolinecarboxamide methanesulfonate.

### Advantageous Effects of Invention

The pharmaceutical composition of the present invention is excellent in dissolution of the quinoline derivative (I), which is a principal agent, and is also excellent in absorption into a living body. The pharmaceutical composition is also a pharmaceutical composition that is maintained even after long term storage.

### BRIEF DESCRIPTION OF DRAWINGS

FIG. 1 shows the dissolution profiles of the compound A from the pharmaceutical compositions obtained in Examples 4 to 6 and Comparative Example 1.

FIG. 2 shows the dissolution profiles of the compound A from the pharmaceutical compositions obtained in Examples 7 to 9 and Comparative Example 2.

FIG. 3 shows the dissolution patterns of the compound A from the pharmaceutical compositions obtained in Examples 10 to 12 and Comparative Example 3.

FIG. 4 shows the dissolution profiles of the compound A from the pharmaceutical compositions obtained in Examples 13 to 15 and Comparative Example 4.

FIG. 5 shows the dissolution profiles of the compound A from the pharmaceutical compositions obtained in Examples 16 to 17 and Comparative Example 5.

FIG. 6 shows the dissolution profiles of the compound A from the pharmaceutical compositions obtained in Example 18 and Comparative Examples 7 to 8.

FIG. 7 shows the dissolution profiles of the compound A from the pharmaceutical compositions obtained in Example 19 and Comparative Examples 9 to 10.

### DESCRIPTION OF EMBODIMENTS

The pharmaceutical composition of the present invention means a composition comprising the quinoline derivative (I) and a basic substance as essential ingredients. A mixing ratio of the quinoline derivative (I) and the basic substance is, but is not limited to, normally 1:0.5 to 50, preferably 1:1 to 25, further preferably 1:2 to 12.5.

In addition, a mixing rate of the quinoline derivative (I) with respect to the total weight of the pharmaceutical composition (excluding a capsule shell) is normally 0.25 to 50 weight %, preferably 0.5 to 25 weight %, further preferably 1 to 12.5 weight %.

A mixing rate of the basic substance with respect to the total weight of the pharmaceutical composition is normally 1 to 60 weight %, preferably 5 to 50 weight %, further preferably 10 to 40 weight %. At least one basic substance of the present invention may be included in the pharmaceutical composition, or two or more basic substances may also be included.

A dosage form of the pharmaceutical composition specifically means a solid preparation such as granules, fine granules, tablets or capsules and so on. It is preferably fine granules, granules or capsules filled with fine granules or granules.

The quinoline derivative (I) is a compound disclosed in WO 2002/32872. A preferable quinoline derivative (I) is a quinoline derivative or pharmacologically acceptable salt thereof or solvate thereof selected from the group consisting of
4-(3-fluoro-4-(cyclopropylaminocarbonyl)aminophenoxy)-7-(2-methoxyethoxy)-6-quinolinecarboxamide,
4-(3-chloro-4-(cyclopropylaminocarbonyl)aminophenoxy)-7-methoxy-6-quinolinecarboxamide,
4-(3-chloro-4-(cyclopropylaminocarbonyl)aminophenoxy)-7-(2-methoxyethoxy)-6-quinolinecarboxamide,
4-(3-chloro-4-(cyclopropylaminocarbonyl)aminophenoxy)-7-(2-hydroxyethoxy)-6-quinolinecarboxamide,
4-(3-chloro-4-(cyclopropylaminocarbonyl)aminophenoxy)-7-((2S)-2,3-dihydroxypropyl)oxy-6-quinolinecarboxamide,
4-(3-chloro-4-(methylaminocarbonyl)aminophenoxy)-7-methoxy-6-quinolinecarboxamide,
4-(3-chloro-4-(ethylaminocarbonyl)aminophenoxy)-7-methoxy-6-quinolinecarboxamide,
N6-methoxy-4-(3-chloro-4-(((ethylamino)carbonyl)amino)phenoxy)-7-methoxy-6-quinolinecarboxamide,
4-(3-chloro-4-(cyclopropylaminocarbonyl)aminophenoxy)-7-(2-ethoxyethoxy)-6-quinolinecarboxamide,
4-(4-((cyclopropylamino)carbonyl)aminophenoxy)-7-(2-methoxyethoxy)-6-quinolinecarboxamide,
N-(2-fluoro-4-[(6-carbamoyl-7-methoxy-4-quinolyl)oxy]phenyl)-N′-cyclopropylurea,
N6-(2-hydroxyethyl)-4-(3-chloro-4-(((cyclopropylamino)carbonyl)amino)phenoxy)-7-methoxy-6-quinolinecarboxamide,
4-(3-chloro-4-(1-propylaminocarbonyl)aminophenoxy)-7-methoxy-6-quinolinecarboxamide,
4-(3-chloro-4-(cis-2-fluoro-cyclopropylaminocarbonyl)aminophenoxy)-7-methoxy-6-quinolinecarboxamide,
N6-methyl-4-(3-chloro-4-(((cyclopropylamino)carbonyl)amino)phenoxy)-7-(2-methoxyethoxy)-6-quinolinecarboxamide and
N6-methyl-4-(3-chloro-4-(((ethylamino)carbonyl)amino)phenoxy)-7-methoxy-6-quinolinecarboxamide.

A more preferable quinoline derivative (I) is a quinoline derivative or pharmacologically acceptable salt thereof or solvate thereof selected from the group consisting of
4-(3-chloro-4-(methylaminocarbonyl)aminophenoxy)-7-methoxy-6-quinolinecarboxamide,
4-(3-chloro-4-(ethylaminocarbonyl)aminophenoxy)-7-methoxy-6-quinolinecarboxamide,
4-(3-chloro-4-(cyclopropylaminocarbonyl)aminophenoxy)-7-methoxy-6-quinolinecarboxamide,
N6-methoxy-4-(3-chloro-4-(((cyclopropylamino)carbonyl)amino)phenoxy)-7-methoxy-6-quinolinecarboxamide and
N6-methoxy-4-(3-chloro-4-(((ethylamino)carbonyl)amino)phenoxy)-7-methoxy-6-quinolinecarboxamide.

A particularly preferable quinoline derivative (I) is 4-(3-chloro-4-(cyclopropylaminocarbonyl)aminophenoxy)-7-methoxy-6-quinolinecarboxamide or pharmacologically acceptable salt thereof or solvate thereof.

The pharmaceutically acceptable salt of the present invention means hydrochloride, hydrobromide, p-toluenesulfonate, sulfate, methanesulfonate or ethanesulfonate. It is preferably the methanesulfonate.

The solvate of the present invention means hydrate, dimethyl sulfoxide solvate or acetic acid solvate.

The quinoline derivative (I) is preferably a crystal of a salt of 4-(3-chloro-4-(cyclopropylaminocarbonyl)aminophenoxy)-7-methoxy-6-quinolinecarboxamide, or a solvate thereof disclosed in WO 2005/063713. A particularly preferred quinoline derivative (I) is the C Form crystal of 4-(3-chloro-4-(cyclopropylaminocarbonyl)aminophenoxy)-7-methoxy-6-quinolinecarboxamide methanesulfonate.

The quinoline derivative (I) is useful as a preventive or therapeutic agent against various tumors and as a metastasis inhibitor against tumors. Examples of the tumors against which the quinoline derivative (I) is effective include thyroid cancer, non-small-cell lung cancer, melanoma, laryngophaiyngeal cancer, esophageal cancer, gastric cancer, colorectal cancer, hepatocellular carcinoma, renal cell carcinoma, pancreatic cancer, bladder cancer, breast cancer, uterine cancer, ovarian cancer, prostate cancer, testicular cancer, gastrointestinal stromal tumor, sarcoma, osteogenic sarcoma, angioma, malignant lymphoma, myeloid leukemia, neuroma and neuroglioma.

The basic substance of the present invention means a basic inorganic salt. Such basic inorganic salts include beryllium carbonate, magnesium carbonate, calcium carbonate, strontium carbonate, barium carbonate, potassium carbonate, calcium hydrogenphosphate and titanium oxide. It is preferably an alkaline earth metal salt of carbonic acid, further preferably magnesium carbonate or calcium carbonate.

It is also acceptable to further include a disintegrating agent in the pharmaceutical composition of the present invention. Such a disintegrating agent include corn starch, partially pregelatinized starch, hydroxypropyl starch, carmellose, carmellose sodium, carmellose calcium, carboxymethyl starch sodium, croscarmellose sodium, low-substituted hydroxypropylcellulose and crospovidone. It is preferably the croscarmellose sodium, the low-substituted hydroxypropylcellulose or the crospovidone.

The pharmaceutical composition of the present invention may be prepared by a known method such as a method described in the General Rules for Preparations in the Japanese Pharmacopoeia Fifteenth Edition.

For example, in the case of the granule, it is possible to add an excipient, a binder, a disintegrating agent, a solvent, or the like to the quinoline derivative (I) as needed, to perform agitation granulation, extruding granulation, tumbling granulation, fluidized-bed granulation, spray granulation, or the like, and to prepare it. It is also acceptable to be coated with an atomizing agent containing the quinoline derivative (I) and an additive such as corn starch, microciystalline cellulose, hydroxypropylcellulose, methylcellulose or polyvinylpyrrolidone while spraying water or a solution of a binder such as saccharose, hydroxypropylcellulose or hydroxypropylmethylcellulose on a core material such as a purified sucrose spherical granule, a lactose/crystalline cellulose spherical granule, a saccharose/starch spherical granule or a granular crystalline cellulose. It is also acceptable to perform sizing and milling as needed.

It is also possible to further, as needed, add an excipient, a binder, a disintegrating agent, a lubricant, an anti-oxidizing agent, a corrigent, a coloring agent, a flavoring agent, or the like to the granule prepared in this way and to compress it to be a tablet. A required excipient may be added to the quinoline derivative (I) to directly compress the mixture into a tablet. It is also possible to fill a capsule with the quinoline derivative (I) added/mixed with an excipient such as lactose, saccharose, glucose, starch, microciystalline cellulose, powdered glycyrrhiza, mannitol, calcium phosphate or calcium sulfate, or with the granule.

Examples of the excipient include lactose, saccharose, glucose, fructose, starch, potato starch, corn starch, wheat starch, rice starch, crystalline cellulose, microciystalline cellulose, powdered glycyrrhiza, mannitol, erythritol, maltitol, sorbitol, trehalose, silicic anhydride, calcium silicate, sodium hydrogencarbonate, calcium phosphate, anhydrous calcium phosphate and calcium sulfate.

Examples of the binder include gelatin, starch, gum arabic, tragacanth, carboxymethylcellulose, hydroxypropylcellulose, hydroxypropylmethylcellulose, polyvinylpyrrolidone, methylcellulose, partially pregelatinized starch, pregelatinized starch, polyvinyl alcohol, sodium arginine, pullulan and glycerin.

Examples of the disintegrating agent include corn starch, partially pregelatinized starch, hydroxypropyl starch, carmellose, carmellose sodium, carmellose calcium, carboxymethyl starch sodium, croscarmellose sodium, low-substituted hydroxypropylcellulose and crospovidone.

Examples of the lubricant include magnesium stearate, stearic acid, calcium stearate, sodium stearyl fumarate, talc and macrogol.

Examples of the anti-oxidizing agent include sodium ascorbate, L-cysteine, sodium sulfite, tocopherol and soybean lecithin.

Examples of the corrigent include citric acid, ascorbic acid, tartaric acid, malic acid, aspartame, acesulfame potassium, thaumatin, saccharin sodium, dipotassium glycyrrhizinate, sodium glutamate, sodium 5-inosinate and sodium 5′-guanylate.

Examples of the coloring agent include titanium oxide, iron sesquioxide, iron sesquioxide yellow, cochineal, carmine, riboflavin, food yellow No. 5 and food blue No. 2.

Examples of the flavoring agent include lemon oil, orange oil, menthol, peppermint oil, bomeol and vanilla flavor.

### EXAMPLES

The present invention will be described in more detail below with reference to Examples, but is not limited to the Examples.

### Examples 1 to 3

Wet granulation was performed with purified water as a solvent using a high-shear granulator (apparatus name: FM-VG-10, manufactured by Powrex Corporation) with the C form crystal of 4-(3-chloro-4-(cyclopropylaminocarbonyl)aminophenoxy)-7-methoxy-6-quinolinecarboxamide methanesulfonate (hereinafter referred to as compound A), D-mannitol (trade name: Mannitol, Merck), precipitated calcium carbonate (trade name: Whiton F, Shiraishi Calcium), hydroxypropylcellulose (HPC-L, Nippon Soda), low-substituted hydroxypropylcellulose (trade name: L-HPC (LH-21), Shin-Etsu Chemical) and microciystalline cellulose (trade name: Ceolus PH-101, Asahi Kasei Chemicals) according to the formulation proportions in Table 1. The granules of which a moisture content was reduced to be less than 2% by further drying were sized using a screen mill (apparatus name: Power Mill P-04S, manufactured by Showa Giken KK) so that their granule diameters were less than 1 mm. Then, microciystalline cellulose (trade name: Ceolus PH-102, Asahi Kasei Chemicals) and talc (trade name: Hi-Filler 17, Iwai Chemicals Company) were added to the sized granules according to the formulation proportions in Table 1, and the mixture was thoroughly mixed using a diffusion (tumbler-type) mixer (trade name: 10 L/20 L Exchange-type Tumbler Mixer, manufactured by Toyo Packing Corporation). Hard capsules size #4 were filled with 100 mg of the resultant granules to prepare capsules containing the compound A.

TABLE 1

| | Ex. 1 | Ex. 2 | Ex. 3 |
|---|---|---|---|
| Compound A | 1.25 | 5 | 12.5 |
| Precipitated calcium carbonate | 33 | 33 | 33 |
| D-Mannitol | 19.75 | 16 | 8.5 |
| Hydroxypropylcellulose | 3 | 3 | 3 |
| Low-substituted hydroxypropylcellulose | 25 | 25 | 25 |
| Microcrystalline cellulose (PH-101) | 10 | 10 | 10 |
| Microcrystalline cellulose (PH-102) | 5 | 5 | 5 |
| Talc | 3 | 3 | 3 |
| Total | 100 | 100 | 100 |

Unit: weight %

### Examples 4 to 9, Comparative Examples 1 to 2

The compound A, precipitated calcium carbonate, low-substituted hydroxypropylcellulose, D-mannitol and talc were thoroughly mixed using a mortar and a pestle according to the formulation proportions in Table 2 and Table 3. Hard capsules size #3 were filled with 100 mg of the resultant mixtures to prepare capsules in Examples 4 to 9. Capsules in Comparative Examples 1 to 2, which contained no precipitated calcium carbonate, were also prepared by the same method.

TABLE 2

| | Com. | | | |
|---|---|---|---|---|
| | Ex. 1 | Ex. 4 | Ex. 5 | Ex. 6 |
| Compound A | 5 | 5 | 5 | 5 |
| Precipitated calcium carbonate | 0 | 5 | 10 | 20 |
| Low-substituted hydroxypropylcellulose | 30 | 25 | 20 | 10 |
| D-Mannitol | 62 | 62 | 62 | 62 |
| Talc | 3 | 3 | 3 | 3 |
| Total | 100 | 100 | 100 | 100 |

Unit: weight %

TABLE 3

| | Com. | | | |
|---|---|---|---|---|
| | Ex. 2 | Ex. 7 | Ex. 8 | Ex. 9 |
| Compound A | 20 | 20 | 20 | 20 |
| Precipitated calcium carbonate | 0 | 5 | 10 | 20 |
| Low-substituted hydroxypropylcellulose | 30 | 25 | 20 | 10 |
| D-Mannitol | 47 | 47 | 47 | 47 |
| Talc | 3 | 3 | 3 | 3 |
| Total | 100 | 100 | 100 | 100 |

Unit: weight %

### Test Example 1

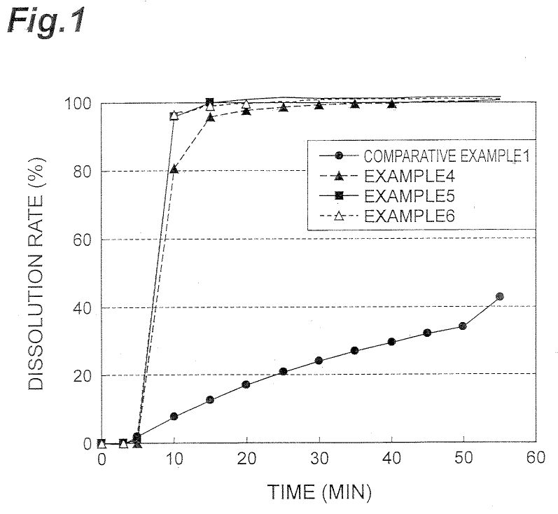

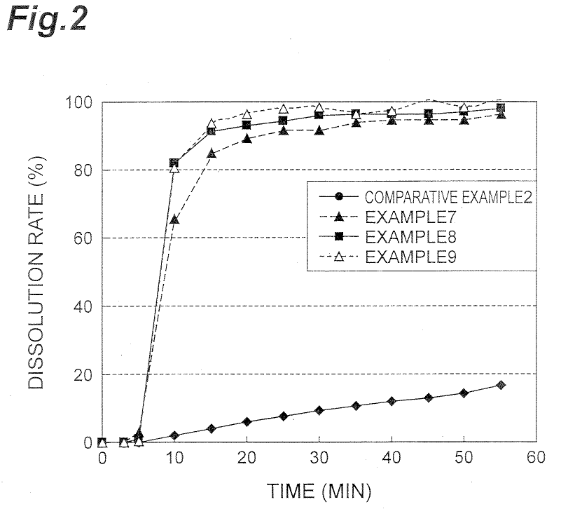

The dissolutions of the compound A in the capsules in Examples 4 to 9 and Comparative Examples 1 to 2 were examined according to the Dissolution Test (the Paddle method, test medium: JP1 solution) described in the Japanese Pharmacopoeia Fifteenth Edition. As a result, the dissolutions of the compound A in the capsules in Comparative Examples 1 to 2, in which no calcium carbonate was mixed, were insufficient. In contrast, the dissolutions of the compound A in the capsules in Examples 4 to 9, in which calcium carbonate was mixed, were good (FIG. 1 and FIG. 2).

### Examples 10 to 15, Comparative Examples 3 to 4

The compound A, magnesium carbonate (Kyowa Chemical Industry), low-substituted hydroxypropylcellulose, D-mannitol and talc were thoroughly mixed using a mortar and a pestle according to the formulation proportions in Table 4 and Table 5. Hard capsules size #3 were filled with 100 mg of the resultant mixtures to prepare capsules in Examples 10 to 15. Capsules in Comparative Examples 3 to 4, which contained no magnesium carbonate, were also prepared by the same method.

TABLE 4

| | Com. | | | |
|---|---|---|---|---|
| | Ex. 3 | Ex. 10 | Ex. 11 | Ex. 12 |
| Compound A | 5 | 5 | 5 | 5 |
| Magnesium carbonate | 0 | 5 | 10 | 20 |
| Low-substituted hydroxypropylcellulose | 30 | 25 | 20 | 10 |
| D-Mannitol | 62 | 62 | 62 | 62 |
| Talc | 3 | 3 | 3 | 3 |
| Total | 100 | 100 | 100 | 100 |

Unit: weight %

TABLE 5

| | Com. | | | |
|---|---|---|---|---|
| | Ex. 4 | Ex. 13 | Ex. 14 | Ex. 15 |
| Compound A | 20 | 20 | 20 | 20 |
| Magnesium carbonate | 0 | 5 | 10 | 20 |
| Low-substituted hydroxypropylcellulose | 30 | 25 | 20 | 10 |
| D-Mannitol | 47 | 47 | 47 | 47 |
| Talc | 3 | 3 | 3 | 3 |
| Total | 100 | 100 | 100 | 100 |

Unit: weight %

### Test Example 2

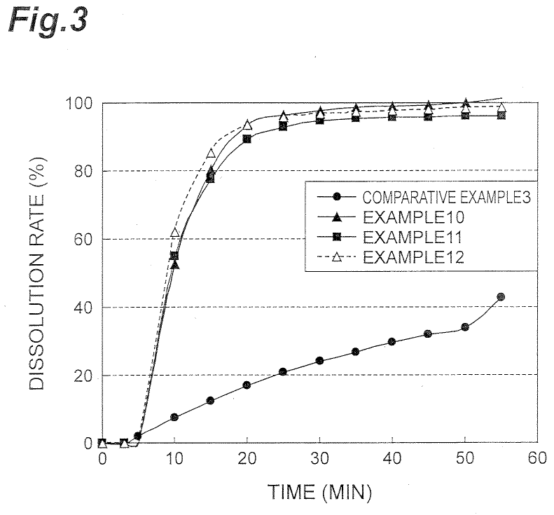

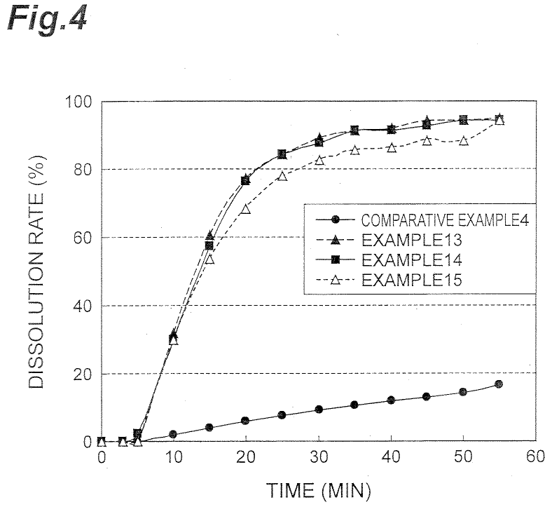

The dissolutions of the compound A in the capsules in Examples 10 to 15 and Comparative Examples 3 to 4 were examined by the same method as in Test Example 1. The dissolutions of the compound A in the capsules in Comparative Examples 3 to 4, in which no magnesium carbonate was mixed, were insufficient. In contrast, the dissolutions of the compound A in the capsules in Examples 10 to 15, in which the magnesium carbonate was mixed, were good (FIG. 3 and FIG. 4).

Examples 16 to 17, Comparative Examples 5 to 6 Purified water was added to the compound A, precipitated calcium carbonate or magnesium carbonate, hydroxypropylcellulose and croscarmellose sodium (trade name: Ac-Di-Sol, Asahi Kasei Chemicals) to perform granulation using a mortar and a pestle, followed by sizing of the dried granules so that their granule diameters were less than 1 mm. Then, microcrystalline cellulose (trade name: Ceolus PH-102, Asahi Kasei Chemicals), low-substituted hydroxypropylcellulose and talc (trade name: Hi-Filler 17, Iwai Chemicals Company) were added to the sized granules according to the formulation proportions in Table 6, and the mixture was mixed thoroughly. Hard capsules size #4 were filled with 100 mg of the resultant mixtures to prepare capsules in Examples 16 to 17. Capsules in Comparative Examples 5 to 6, which contained neither precipitated calcium carbonate nor magnesium carbonate but contained mannitol or talc as a substitute, were also similarly prepared according to the formulation proportions in Table 7.

TABLE 6

| | Ex. 16 | Ex. 17 |
|---|---|---|
| Compound A | 10 | 10 |
| Precipitated calcium carbonate | 15 | 0 |
| Magnesium carbonate | 0 | 15 |
| Hydroxypropylcellulose | 2 | 2 |
| Croscarmellose sodium | 10 | 10 |
| Low-substituted hydroxypropylcellulose | 20 | 20 |
| Microcrystalline cellulose (PH-102) | 41 | 41 |
| Talc | 2 | 2 |
| Total | 100 | 100 |

Unit: weight %

TABLE 7

| | Com. | Com. |
|---|---|---|
| | Ex. 5 | Ex. 6 |
| Compound A | 10 | 10 |
| Mannitol | 15 | 0 |
| Talc | 0 | 15 |
| Hydroxypropylcellulose | 2 | 2 |
| Croscarmellose sodium | 10 | 10 |
| Low-substituted hydroxypropylcellulose | 20 | 20 |
| Microcrystalline cellulose (PH-102) | 41 | 41 |
| Talc | 2 | 2 |
| Total | 100 | 100 |

Unit: weight %

### Test Example 3

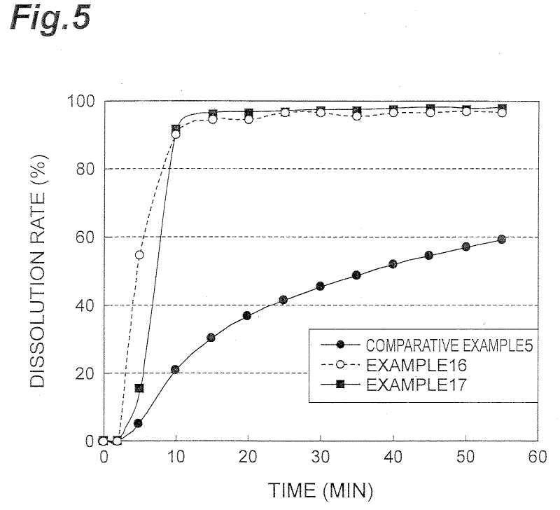

The dissolutions of the compound A in the capsules in Examples 16 to 17 and Comparative Example 5 were examined by the same method as in Test Example 1. The dissolution of the compound A in the capsule in Comparative Example 5, in which neither calcium carbonate nor magnesium carbonate was mixed, was insufficient. In contrast, the dissolutions of the compound A in the capsules in Examples 16 to 17, in which calcium carbonate or magnesium carbonate was mixed, were good (FIG. 5).

### Test Example 4

The capsules in Examples 16 to 17 and Comparative Example 6 were stored for 1 week in an open system under an environment at a temperature of 60° C. and a relative humidity of 75%, followed by determining the production of the degradants with high-performance liquid chromatography. In the capsule formulation in Comparative Example 6, in which neither calcium carbonate nor magnesium carbonate was mixed, an amount of the degradants was increased. In contrast, in the capsules in Examples 16 to 17, in which calcium carbonate or magnesium carbonate was mixed, no increase in amount of the degradants was observed (Table 8).

TABLE 8

| | | Quantitated |
|---|---|---|
| | Degradants | compound A |
| | (%) | (%) |
| Compound A (Initial) | 1.61% | 98.38% |
| Com. Ex. 6 | 1.92% | 98.08% |
| Ex. 16 | 1.50% | 98.50% |
| Ex. 17 | 1.57% | 98.44% |

### Examples 18 to 19, Comparative Examples 7 to 10

The respective ingredients were mixed according to the formulations of Tables 9 and 10 by the same method as in Examples 4 to 9 and Comparative Examples 1 to 2. Hard capsules size #3 were filled with 100 mg of the resultant mixtures to prepare capsules in Examples 18 to 19 and Comparative Examples 7 to 10.

TABLE 9

| | | Com. | Com. |
|---|---|---|---|
| | Ex. 18 | Ex. 7 | Ex. 8 |
| Compound A | 20 | 20 | 20 |
| Precipitated calcium carbonate | 10 | 0 | 0 |
| Calcium oxide | 0 | 10 | 0 |
| Calcium hydroxide | 0 | 0 | 10 |
| Low-substituted hydroxypropylcellulose | 20 | 20 | 20 |
| D-Mannitol | 47 | 47 | 47 |
| Talc | 3 | 3 | 3 |
| Total | 100 | 100 | 100 |

Unit: weight %

TABLE 10

| | | Com. | Com. |
|---|---|---|---|
| | Ex. 19 | Ex. 9 | Ex. 10 |
| Compound A | 20 | 20 | 20 |
| Magnesium carbonate | 10 | 0 | 0 |
| Magnesium oxide | 0 | 10 | 0 |
| Magnesium hydroxide | 0 | 0 | 10 |
| Low-substituted hydroxypropylcellulose | 20 | 20 | 20 |
| D-Mannitol | 47 | 47 | 47 |
| Talc | 3 | 3 | 3 |
| Total | 100 | 100 | 100 |

Unit: weight %

### Test Example 5

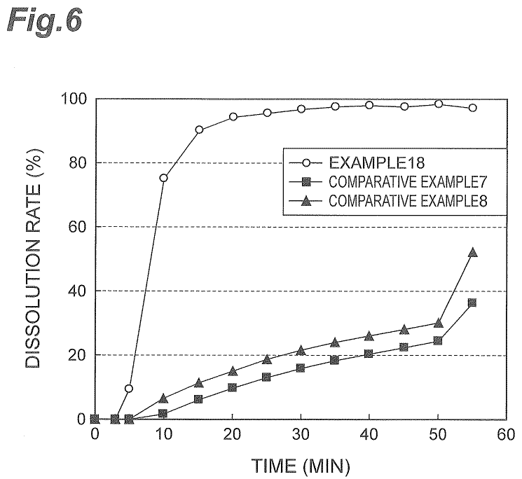

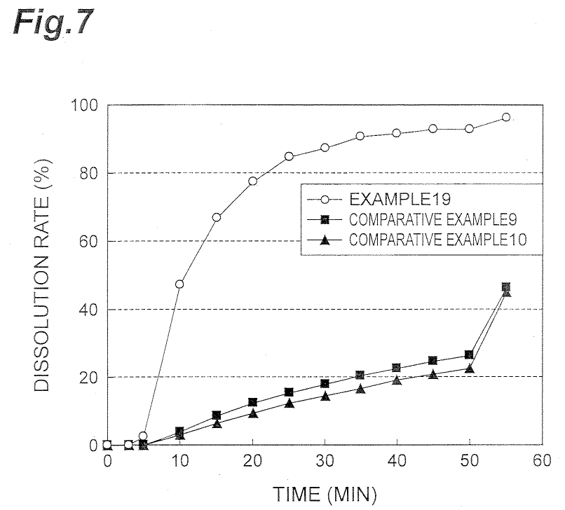

The dissolutions of the compound A in the capsules in Examples 18 to 19 and Comparative Examples 7 to 10 were examined by the same method as in Test Example 1. As a result, the dissolutions of the compound A in the capsules in Comparative Examples 7 to 10, in which calcium oxide, calcium hydroxide, magnesium oxide or magnesium hydroxide was mixed, were insufficient. In contrast, the dissolutions of the compound A in the capsules in Examples 18 to 19, in which calcium carbonate or magnesium carbonate was mixed, were good (FIG. 6 and FIG. 7).

### INDUSTRIAL APPLICABILITY

The pharmaceutical composition of the present invention is excellent in dissolution of the quinoline derivative and also in stability, and is therefore useful as a medicament for prevention or treatment of a tumor.

1-12. (canceled)

13. A pharmaceutical composition, comprising:
(1) 4-(3-chloro-4-(cyclopropylaminocarbonyl)aminophenoxy)-7-methoxy-6-quinolinecarboxamide or a pharmaceutically acceptable salt thereof;
(2) an alkaline earth metal carbonate; and
(3) a disintegrating agent,
wherein the alkaline earth metal carbonate is magnesium carbonate or calcium carbonate.

14. The composition of claim 13, wherein the alkaline earth metal carbonate is magnesium carbonate.

15. The composition of claim 13, wherein the alkaline earth metal carbonate is calcium carbonate.

16. The composition of claim 14, wherein the disintegrating agent is carmellose sodium, carmellose calcium, carboxymethyl starch sodium, croscarmellose sodium, low-substituted hydroxypropylcellulose, or crospovidone.

17. The composition of claim 16, wherein the disintegrating agent is low-substituted hydroxypropylcellulose.

18. The composition of claim 14, wherein the weight ratio of 4-(3-chloro-4-(cyclopropylaminocarbonyl)aminophenoxy)-7-methoxy-6-quinolinecarboxamide or the pharmaceutically acceptable salt thereof to the magnesium carbonate is from 1:1 to 1:25.

19. The composition of claim 14, wherein 4-(3-chloro-4-(cyclopropylaminocarbonyl)aminophenoxy)-7-methoxy-6-quinolinecarboxamide or the pharmaceutically acceptable salt thereof is present in an amount from 1 wt % to 12.5 wt %.

20. The composition of claim 14, wherein the composition is in a capsule dosage form.

21. The composition of claim 14, wherein the composition is in a tablet dosage form.

22. The composition of claim 14, wherein the composition is in a granular form.

23. The composition of claim 15, wherein the disintegrating agent is carmellose sodium, carmellose calcium, carboxymethyl starch sodium, croscarmellose sodium, low-substituted hydroxypropylcellulose, or crospovidone.

24. The composition of claim 23, wherein the disintegrating agent is low-substituted hydroxypropylcellulose.

25. The composition of claim 24, wherein the composition further comprises mannitol, hydroxypropylcellulose, microcrystalline cellulose, and talc.

26. The composition of claim 15, wherein the weight ratio of 4-(3-chloro-4-(cyclopropylaminocarbonyl)aminophenoxy)-7-methoxy-6-quinolinecarboxamide or the pharmaceutically acceptable salt thereof to the calcium carbonate is from 1:1 to 1:25.

27. The composition of claim 15, wherein 4-(3-chloro-4-(cyclopropylaminocarbonyl)aminophenoxy)-7-methoxy-6-quinolinecarboxamide or the pharmaceutically acceptable salt thereof is present in an amount from 1 wt % to 12.5 wt %.

28. The composition of claim 27, wherein the calcium carbonate is present in an amount from 10 wt % to 40 wt %.

29. The composition of claim 15, wherein the composition is in a capsule dosage form.

30. The composition of claim 23, wherein the composition is in a capsule dosage form.

31. The composition of claim 24, wherein the composition is in a capsule dosage form.

32. The composition of claim 25, wherein the composition is in a capsule dosage form.

33. The composition of claim 26, wherein the composition is in a capsule dosage form.

34. The composition of claim 27, wherein the composition is in a capsule dosage form.

35. The composition of claim 28, wherein the composition is in a capsule dosage form.

36. The composition of claim 15, wherein the composition is in a tablet dosage form.

37. The composition of claim 23, wherein the composition is in a tablet dosage form.

38. The composition of claim 24, wherein the composition is in a tablet dosage form.

39. The composition of claim 15, wherein the composition is in a granular form.
